(19) United States
(12) Patent Application Publication
(10) Pub. No.: US 2025/0366720 A1
(43) Pub. Date: Dec. 4, 2025

(54) INFORMATION PROCESSING DEVICE, MOVABLE BODY, AND RECORDING MEDIUM

(71) Applicant: TOYOTA BOSHOKU KABUSHIKI KAISHA, Aichi (JP)

(72) Inventors: Tomoya KITAZAKI, Aichi (JP); Hiroyuki YAMASHIRO, Kyoto (JP)

(73) Assignee: TOYOTA BOSHOKU KABUSHIKI KAISHA, Aichi (JP)

(21) Appl. No.: 19/198,330

(22) Filed: May 5, 2025

(30) Foreign Application Priority Data
May 28, 2024 (JP) 2024-086525

(51) Int. Cl. A61B 5/02; A61B 5/00; A61B 5/11; B60N 2/00

(52) U.S. Cl. CPC A61B 5/02042; A61B 5/1102; A61B 5/6893; A61B 5/7275; A61B 5/747; B60N 2/0022; A61B 2560/0214

(57) ABSTRACT

An information processing device includes an obtaining section that obtains a detection signal from a sensor which is disposed in a seat of a movable body and which detects vibration transmitted to the seat from a passenger seated on the seat and an inferring section that infers a bleeding volume of the passenger on the basis of the detection signal obtained by the obtaining section.

This Nonprovisional application claims priority under 35 U.S.C. § 119 on Patent Application No. 2024-086525 filed in Japan on May 28, 2024, the entire contents of which are hereby incorporated by reference.

### TECHNICAL FIELD

The present disclosure relates to an information processing device, a movable body, and a recording medium.

### BACKGROUND ART

Patent Literature 1 discloses a passenger damage inferring server that inputs video data received from an imaging device that images a cabin of a vehicle, to an inference model to infer damage to the passenger caused by a vehicle accident.

### CITATION LIST

### Patent Literature

### [Patent Literature 1]

Japanese Patent Application Publication Tokukai No. 2020-177444

### SUMMARY OF INVENTION

### Technical Problem

However, in some cases, it is difficult to make inference from video data in accident sites in which movable bodies are involved, such as a case where a passenger is internally bleeding. It is an object of an aspect of the present disclosure to provide an information processing device that makes it possible to appropriately infer a bleeding volume of a passenger of a movable body.

### Solution to Problem

In order to attain the foregoing object, an information processing device in accordance with an aspect of the present disclosure includes: an obtaining section that obtains a detection signal from a sensor which is disposed in a seat of a movable body and which detects vibration transmitted to the seat from a passenger seated on the seat; and an inferring section that infers a bleeding volume of the passenger on the basis of the detection signal obtained by the obtaining section.

The information processing device in accordance with each aspect of the present disclosure may be realized by a computer. In this case, the scope of the present disclosure also includes (i) a program for an information processing device which program causes the computer to realize the information processing device by causing the computer to operate as each section (software element) included in the information processing device and (ii) a computer-readable recording medium storing the program.

### Advantageous Effects of Invention

An aspect of the present disclosure makes it possible to appropriately infer a bleeding volume of a passenger of a movable body.

### BRIEF DESCRIPTION OF DRAWINGS

FIG. 1 is a view illustrating an example of an internal structure of a movable body provided with an information processing device in accordance with Embodiment 1 of the present disclosure.

FIG. 2 is a view used for explaining a position in which a sensor is disposed.

FIG. 3 is a view illustrating one configuration example of the information processing device in accordance with Embodiment 1 of the present disclosure.

FIG. 4 is a view used for explaining a method for inferring a bleeding volume on the basis of a heart rate and a respiratory rate.

FIG. 5 is a view used for explaining a process by a determining section illustrated in FIG. 3.

FIG. 6 is a flowchart illustrating a flow of a process by an information processing device in accordance with an embodiment of the present disclosure.

FIG. 7 is a flowchart illustrating a flow of a process of inferring an urgency level of injury of the passenger which is carried out by an inferring section.

### DESCRIPTION OF EMBODIMENTS

### Embodiment 1

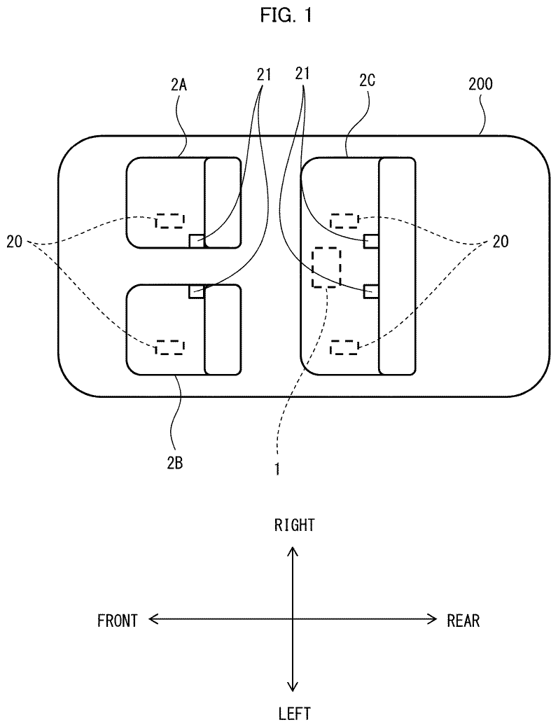

FIG. 1 is a view illustrating an example of an internal structure of a movable body provided with an information processing device in accordance with Embodiment 1 of the present disclosure. Hereinafter, a front-and-rear direction and a left-and-right direction are defined as shown by the arrows in FIG. 1. A movable body 200 illustrated in FIG. 1 is an automobile and includes, in a vehicle thereof, seats 2A, 2B, and 2C on which passengers can be seated. The seat 2A is a driver seat of the movable body 200, the seat 2B is a front passenger seat of the movable body 200, and the seat 2C is a rear seat of the movable body 200 on which two passengers can be seated.

An information processing device 1 is provided in the movable body 200. The information processing device 1 is disposed in a position that is unlikely to be affected by an accident, such as a collision accident. In FIG. 1, the information processing device 1 is disposed under the seat 2C.

In the seats 2A, 2B, and 2C illustrated in FIG. 1, in respective seating positions thereof, sensors 20 and wear detecting sections 21 are disposed. In each of the seats 2A and 2B, the sensor 20 is disposed in a position closer to the left than the center of the seating face, and one wear detecting section 21 is disposed in a position of a buckle of a seatbelt. In the seat 2C having two seating positions, one sensor 20 is disposed, in each seating position, in a position closer to the left than the center of the seating face. Further, in the seat 2C, one wear detecting section 21 is disposed in a position of a buckle of a seatbelt for each seating position.

The sensor 20 detects vibration transmitted from the seated passenger to the seating face and outputs a detection signal corresponding to the vibration detected. For example, the sensor 20 is a piezoelectric sensor that detects ballistic movement of the heart of the seated passenger.

The wear detecting section 21 detects that the passenger is wearing a seatbelt. For example, in a case where a tongue of the seatbelt is inserted into a buckle of the seatbelt, the wear detecting section 21 detects that the passenger is wearing the seatbelt.

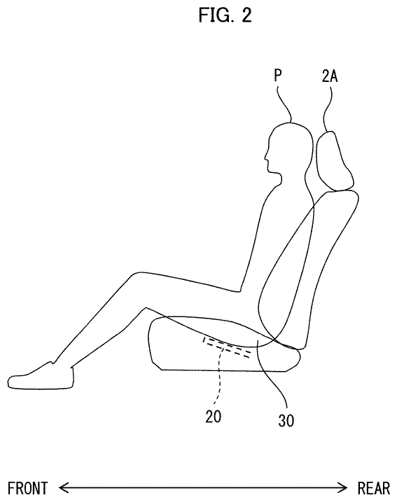

FIG. 2 is a view used for explaining a position in which a sensor is disposed. As illustrated in FIG. 2, the sensor 20 is disposed beneath the seating face of the seat 2A and is located in the vicinity of the ischium 30 of a passenger P seated on the seat 2A. Disposing the sensor 20 beneath the seating face enables the sensor 20 to detect the vibration transmitted from the passenger P to the seating face, even if a collision accident in which the movable body 200 is involved causes the passenger P to tilt forward due to an inertial force.

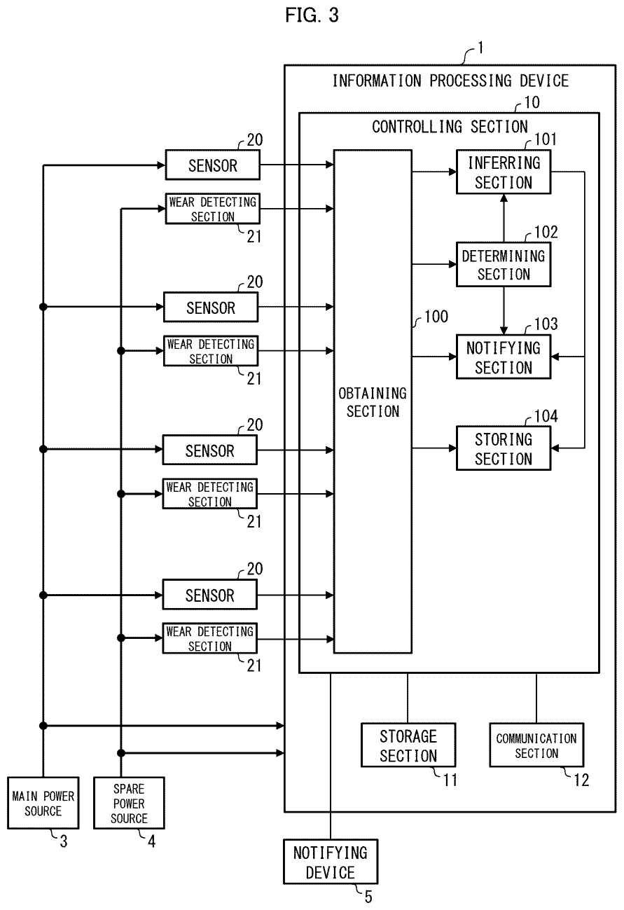

FIG. 3 is a view illustrating one configuration example of the information processing device in accordance with Embodiment 1 of the present disclosure. As illustrated in FIG. 3, the information processing device 1 includes a controlling section 10, a storage section 11, and a communication section 12. The controlling section 10 includes, for example, a central processing unit (CPU) and a random access memory (RAM). The storage section 11 includes, for example, a hard disk drive (HDD) or a solid state drive (SSD). The storage section 11 may be a non-volatile storage medium that is configured to be detachably attached to the information processing device 1. The storage section 11 stores a program to be executed by the controlling section 10. The communication section 12 is, for example, a communication module that connects to a mobile phone network.

A main power source 3 and a spare power source 4 are provided in the movable body 200 and each supply electric power to the information processing device 1, the sensor 20, and the wear detecting section 21. The main power source 3 is one example of a first power source. The main power source 3 is, for example, a battery provided in the movable body 200, and is used for driving electric components of the movable body 200. The spare power source 4 is one example of a second power source. When the main power source 3 malfunctions due to an accident, the spare power source 4 supplies electric power to the information processing device 1, the sensor 20, and the wear detecting section 21. The spare power source 4 is disposed in a position that is unlikely to be affected by an accident, such as a collision accident, e.g., beneath the seat 2C or the like. The spare power source 4 has a capacity capable of continuing supplying electric power at least until, for example, an ambulance arrives at the accident site.

The information processing device 1 is connected to a notifying device 5. Examples of the notifying device 5 include a display device, a speaker, and a lighting device. Examples of the display device include a monitor of a navigation device, a monitor for providing video content disposed in the movable body 200, and an information terminal possessed by the passenger P. Examples of the lighting device include headlamps, brake lamps, turn signal lamps, and hazard lamps.

The controlling section 10 carries out a program stored in, for example, the storage section 11 to function as an obtaining section 100, an inferring section 101, a determining section 102, a notifying section 103, and a storing section 104.

The obtaining section 100 obtains a detection signal from the sensor 20 every predetermined time. The predetermined time is, for example, 2 seconds. The predetermined time may be changed in accordance with a state of the movable body 200. For example, after the movable body 200 has been involved in a collision accident, the predetermined time may be shorter. The obtaining section 100 may obtain, from the wear detecting section 21, a detection signal indicating that the passenger is wearing a seatbelt. The detection signal obtained by the obtaining section 100 is inputted to the inferring section 101, the determining section 102, the notifying section 103, and the storing section 104.

The inferring section 101 infers a bleeding volume of the passenger P on the basis of the detection signal obtained by the obtaining section 100. More specifically, the inferring section 101 infers a heart rate and a respiratory rate of the passenger P on the basis of the detection signal obtained by the obtaining section 100 and infers a bleeding volume of the passenger P on the basis of a result of the inference of the heart rate and the respiratory rate of the passenger P. Since a method for inferring a heart rate of the passenger P from a ballistic movement of the heart of the passenger P detected by the sensor 20 has been known, a detailed description thereof will be omitted here.

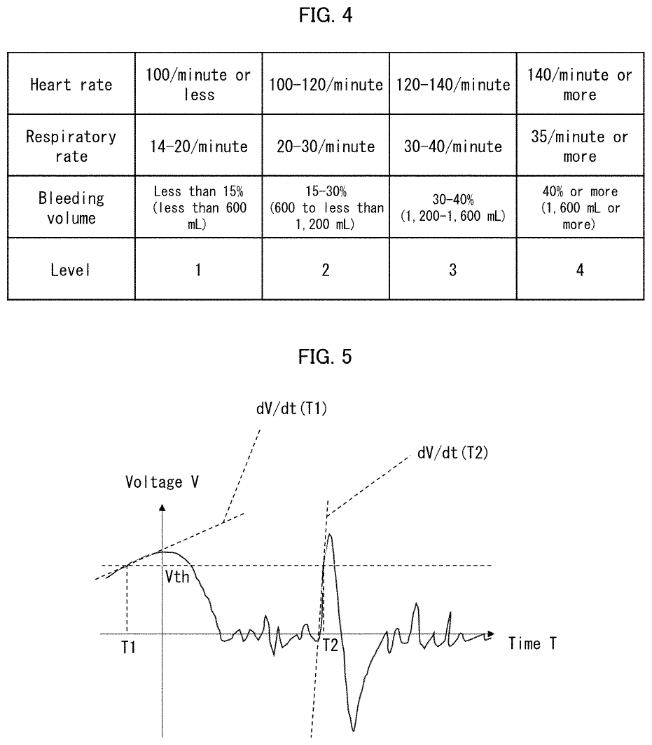

FIG. 4 is a view used for explaining a method for inferring a bleeding volume on the basis of a heart rate and a respiratory rate. As illustrated in FIG. 4, for example, in a case where the heart rate per minute of the passenger P is not more than 100 times and the respiratory rate per minute of the passenger P is not more than 14 to 20 times, the inferring section 101 infers that the bleeding volume of the passenger P is less than 15% of the blood volume of the passenger P (level 1).

A circulating blood volume of a human is said to be approximately 1/12 (men) to 1/13 (women) of a body weight. In a case where the body weight of the passenger P is 60 kg, the blood volume of the passenger P is approximately 4 L to 5 L. In this case, the bleeding volume of 15% of the blood volume is approximately 600 mL to 750 mL.

For example, in a case where the heart rate per minute of the passenger P is in a range of 100 to 120 times and the respiratory rate per minute of the passenger P is in a range of 20 to 30 times, the inferring section 101 infers that the bleeding volume of the passenger P is 15% to 30% of the blood volume of the passenger P (level 2).

For example, in a case where the heart rate per minute of the passenger P is in a range of 120 to 140 times and the respiratory rate per minute of the passenger P is in a range of 30 to 40 times, the inferring section 101 infers that the bleeding volume of the passenger P is 30% to 40% of the blood volume of the passenger P (level 3).

For example, in a case where the heart rate per minute of the passenger P is not less than 140 times and the respiratory rate per minute of the passenger P is not less than 35 times, the inferring section 101 infers that the bleeding volume of the passenger P is not less than 40% of the blood volume of the passenger P (level 4).

In a case where the inference result based on the heart rate per minute of the passenger P is different from the inference result based on the respiratory rate per minute of the passenger P, the inferring section 101 may obtain, as the inferred value, one exhibiting a larger inferred value of the bleeding volume of the passenger P. Alternatively, the inferring section 101 may set, as the inferred value of the bleeding volume of the passenger P, an average value of the inference result based on the heart rate per minute of the passenger P and the inference result based on the respiratory rate per minute of the passenger P.

The inferring section 101 infers an urgency level of injury of the passenger P on the basis of a result of inference of the bleeding volume, the heart rate, and the respiratory rate of the passenger P. The urgency level of injury of the passenger P is, for example, inferred in four levels: urgency levels 0 (the most urgent), I (urgent), II (semi-urgent), and III (standby). The process in which the inferring section 101 infers an urgency level of injury of the passenger P will be described later.

The determining section 102 in FIG. 3 determines whether or not the movable body 200 has been involved in a collision accident, on the basis of the detection signal obtained by the obtaining section 100. FIG. 5 is a view used for explaining a process by a determining section illustrated in FIG. 3. FIG. 5 illustrates a change over time in a voltage V of the detection signal obtained by the obtaining section 100 from the sensor 20. The passenger P has seated on the seat 2A immediately before the time T1, and thus the absolute value of the detection signal from the sensor 20 is then gradually increased in FIG. 5. The movable body 200 is involved in a collision accident immediately before the time T2, and thus the absolute value of the detection signal from the sensor 20 is then steeply increased.

For example, when the detection signal obtained by the obtaining section 100 from the sensor 20 satisfies both the following determination conditions (1) and (2), the determining section 102 determines that the movable body 200 has been involved in a collision accident.
Condition (1): An absolute value |V(t)| of the detection signal obtained by the obtaining section 100 from any of the sensors 20 disposed in the movable body 200 is not less than a first threshold Vth.
Condition (2): An inclination dV/dt of a change over time in the detection signal obtained by the obtaining section 100 from any of the sensors 20 disposed in the movable body 200 is not less than a second threshold.

The first threshold is set at a level not exceeded under a normal ballistic movement of the heart of the passenger P, and may be exceeded when the passenger P moves his/her body. The first threshold is set in advance in a design phase of the information processing device 1. In FIG. 5, the absolute value IV(t) of the voltage of the detection signal becomes not less than the first threshold Vth at the time T1 and at the time T2.

The second threshold is set at a level not exceeded under the vibration caused when the passenger P has seated, and is set in advance in the design phase of the information processing device 1. That is, in FIG. 5, the inclination dV/dt (T1) of the change over time in the voltage of the detection signal at the time T1 is less than the second threshold, and dV/dt (T2) is not less than the second threshold.

For example, at the time T1, since the determination condition (2) is not satisfied, the determining section 102 determines that the movable body 200 is not involved in a collision accident. Meanwhile, at the time T2, since both the determination conditions (1) and (2) are satisfied, the determining section 102 determines that the movable body 200 has been involved in a collision accident.

The determining section 102 may further use a signal obtained by the obtaining section 100 from the wear detecting section 21 for the determination of whether or not the movable body 200 has been involved in a collision accident. For example, the determining section 102 may use, for the determination, only a detection signal obtained from a sensor 20 disposed in a seat or seating position in which the seatbelt is worn, among the sensors 20 disposed in the movable body 200.

The notifying section 103 in FIG. 3 notifies, via the notifying device 5, an urgency level of injury of the passenger P inferred by the inferring section 101, to an inside and outside of the movable body 200. In a case where the notification is made with use of the speaker of the movable body 200 as the notifying device 5, the notifying section 103 may output a voice corresponding to the inference result of the urgency level of the injury of the passenger P inferred by the inferring section 101 in a volume that is audible outside the movable body 200. In a case where making the notification using the speaker, the notifying section 103 may give a simple instruction to the passenger P to check a consciousness level of the passenger P.

In a case where a sequential winker, which is one type of turn signal lamp, is used as the notifying device 5 for the notification, the notifying section 103 may turn on a number of lamps corresponding to the urgency level of the injury of the passenger P. For example, in a case where the urgency level is III (standby), one lamp may be turned on, in a case where the urgency level is II (semi-urgent), two lamps may be turned on, in a case where the urgency level is I (urgent), three lamps may be turned on, and in a case where the urgency level is 0 (the most urgent), four lamps may be turned on.

In a case where headlights and tail lights are used as the notifying device 5 for the notification, the notifying section 103 may turn on the light corresponding to the seating position of the passenger P in a manner corresponding to the urgency level of the injury of the passenger P. For example, with respect to a passenger P seated on the seat 2A, the right headlight of the movable body 200 may be caused to blink a number of times corresponding to the urgency level of the injury of the passenger P. With respect to a passenger P seated on the seat 2B, the left headlight of the movable body 200 may be caused to blink a number of times corresponding to the urgency level of the injury of the passenger P. With respect to a passenger P seated on the seat 2C, the tail light corresponding to the seating position of the passenger P may be caused to blink a number of times corresponding to the urgency level of the injury of the passenger P seated on the seating position. The notifying section 103 may notify an urgency level of injury of a passenger P with a high urgency level of the injury among the passengers P of the movable body 200.

The notifying section 103 may carry out the notification with use of a lamp special for notification as the notifying device 5 and may emit light in a color corresponding to the urgency level of the injury of the passenger P. For example, the color indicating the urgency level I (urgent) is red. The color indicating the urgency level II (semi-urgent) is yellow. The color indicating the urgency level III (standby) is green. The color indicating the urgency level 0 (the most urgent) is black. The light indicating the urgency level may be expressed, for example, by applying a color filter to white light to color the light. Black may be expressed by turning off the light. The lamp special for notification may be disposed outside the movable body 200 or may be disposed in each seating position. It is possible to make the notification on all the passengers P seated in the movable body 200 or make the notification on the passenger P with the most serious urgency level of the injury.

In a case where the determining section 102 determines that the movable body 200 has been involved in a collision accident, the storing section 104 stores, in the storage section 11, data on, for example, the detection signal that has been obtained by the obtaining section 100 after a timing a predetermined time before the timing at which the accident occurred. The data stored in the storage section 11 by the storing section 104 is not limited to the detection signal obtained by the obtaining section 100, but, for example, the inference result from the inferring section 101, the determination result from the determining section 102, the information on an operation amount of an operation member of the movable body 200, and an image captured by a camera provided to the movable body 200 may be stored. Examples of the operation member of the movable body 200 include an accelerator pedal, a brake pedal, and a steering wheel. Examples of the camera provided in the movable body 200 include a camera that images the surroundings of the movable body 200 and a camera that images an interior space in which the passenger P of the movable body 200 is seated.

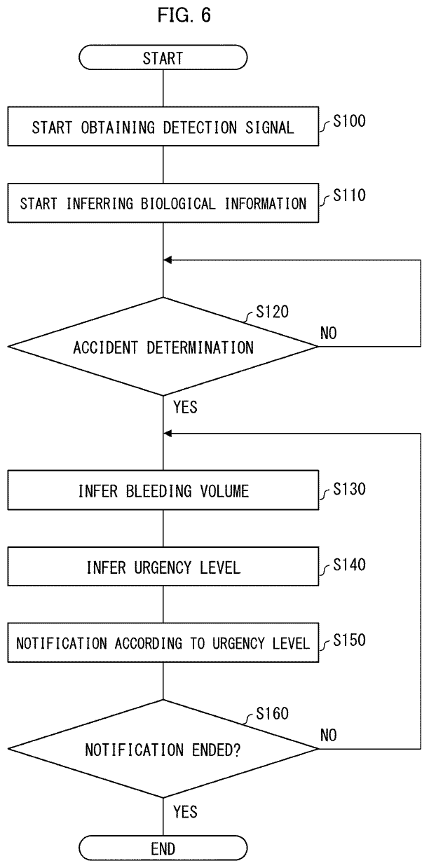

FIG. 6 is a flowchart illustrating a flow of a process by an information processing device in accordance with an embodiment of the present disclosure. On start of supplying of electric power to the information processing device 1, the controlling section 10 of the information processing device 1 starts the process illustrated in FIG. 6.

The controlling section 10 of the information processing device 1 determines whether or not electric power is supplied normally from the main power source 3 while the process in FIG. 6 is being carried out. When the supplying of electric power from the main power source 3 is lost due to, for example, involvement of the movable body 200 in an accident, the controlling section 10 switches to the supplying of electric power from the spare power source 4. In a case where even the supplying of electric power from the spare power source 4 is lost, the controlling section 10 ends the process in FIG. 6.

In S100, the controlling section 10 functions as the obtaining section 100 and starts obtaining the detection signal. Thereafter, the controlling section 10 obtains the detection signal from the sensor 20 and the wear detecting section 21 disposed in each seating position of the movable body 200 every predetermined time. In subsequent S110, the controlling section 10 functions as the inferring section 101 and starts inferring the biological information of the passenger P on the basis of the detection signal obtained by the obtaining section 100.

In subsequent S120, the controlling section 10 functions as the determining section 102 and determines whether or not the movable body 200 has been involved in a collision accident, on the basis of the detection signal obtained by the obtaining section 100. For example, in a case where the detection signal that has been obtained by the obtaining section 100 does not satisfy both the determination conditions (1) and (2) (S120: NO), the controlling section 10 carries out the determination in S120 on the basis of a new detection signal obtained by the obtaining section 100. For example, in a case where it is determined that the detection signal that has been obtained by the obtaining section 100 satisfies both the determination conditions (1) and (2), the controlling section 10 determines that the movable body 200 has been involved in a collision accident (S120: YES) and proceeds to the process in S130.

In subsequent S130, the controlling section 10 functions as the inferring section 101 and infers a heart rate and a respiratory rate of the passenger P on the basis of the detection signal obtained by the obtaining section 100. On the basis of a result of the inference, the controlling section 10 infers a bleeding volume of the passenger P. For example, the controlling section 10 infers a bleeding volume of the passenger P on the basis of the inference result of the heart rate and the respiratory rate of the passenger P, as illustrated in FIG. 4.

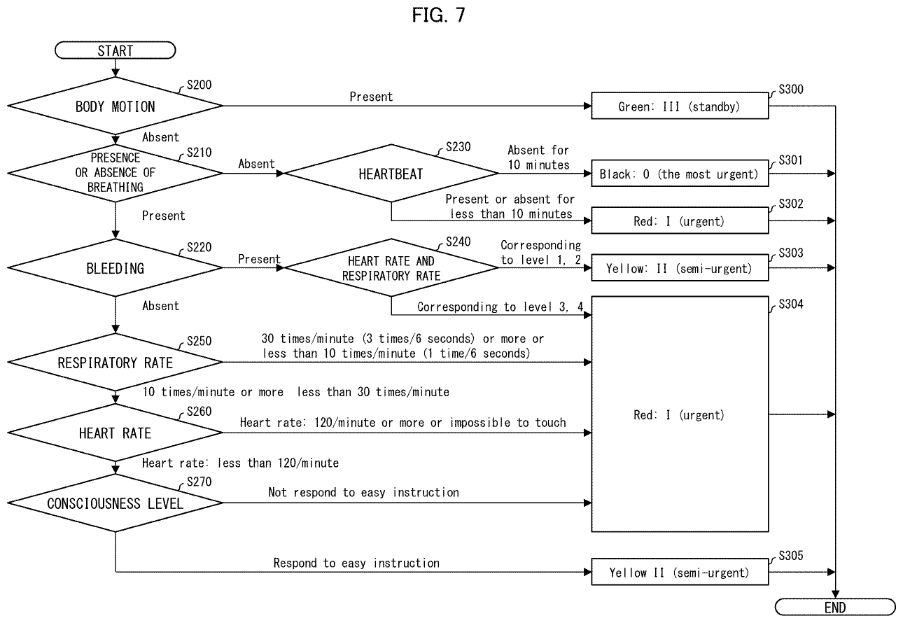

In subsequent S140, the controlling section 10 functions as the inferring section 101 and infers an urgency level of injury of the passenger P on the basis of the inference result of the bleeding volume, the heart rate, and the respiratory rate of the passenger P. One example of the process carried out in S140 will be described later with reference to FIG. 7.

In subsequent S150, the controlling section 10 functions as the notifying section 103 and carries out, via the notifying device 5, notification based on the inference result of the urgency level of the injury of the passenger P which has been inferred in S140. Thus, with respect to the passenger P of the movable body 200 involved in a collision accident, the urgency level of the injury of the passenger P is notified to an inside and outside the movable body 200.

In subsequent S160, the controlling section 10 determines whether to end the notification carried out in S150. For example, in a case where a predetermined operation is carried out by the passenger P of the movable body 200 or an emergency crew who has arrived at the accident site, it is determined to end the notification (S160: YES), and the process in FIG. 6 is ended. In a case where the predetermined operation is not carried out, the controlling section 10 determines that the notification is continued (S160: NO) and proceeds to the process in S130.

FIG. 7 is a flowchart illustrating a flow of a process of inferring an urgency level of injury of the passenger carried out by the inferring section. The process by the inferring section 101 which is illustrated in FIG. 7 is carried out for each passenger P of the movable body 200 in, for example, S140 of FIG. 6.

In S200, the controlling section 10 determines presence or absence of body motion of the passenger P. For example, the controlling section 10 determines that the body movement is present for the passenger P whose absolute value IV(t) I of the detection signal obtained by the obtaining section 100 from the sensor 20 is not less than the first threshold Vth. The controlling section 10 may determine the presence or absence of the body motion of the passenger P on the basis of an image obtained by imaging the passenger P with use of an imaging device, such as a camera. With respect to a passenger P with body motion (S200: present), the controlling section 10 determines that the urgency level of the injury of the passenger P is “green: III (standby)” (S300). With respect to a passenger P without body motion (S200: No), the controlling section 10 proceeds to the process in S210.

In S210, the controlling section 10 determines whether or not the passenger P is breathing. The controlling section 10 determines whether or not the passenger P is breathing, for example, on the basis of an inferred value of the respiratory rate of the passenger P which has been inferred by the inferring section 101. For example, in a case where the inferred value of the respiratory rate of the passenger P inferred by the inferring section 101 is less than 14 times/minute, the controlling section 10 determines that the passenger P is not breathing. With respect to the passenger P determined to be breathing (S210: present), the controlling section 10 proceeds to the process in S220. With respect to the passenger P determined to be not breathing (S210: absent), the controlling section 10 proceeds to the process in S230.

In S220, the controlling section 10 determines whether or not the passenger P is bleeding. The controlling section 10 determines whether or not the passenger P is bleeding, for example, on the basis of an inferred value of the bleeding volume of the passenger P which has been inferred by the inferring section 101. For example, in a case where the inferred value of the bleeding volume of the passenger P which has been inferred by the inferring section 101 is less than 15% of the body weight of the passenger P, the controlling section 10 may determine that the passenger P is not bleeding. With respect to the passenger P determined to be bleeding (S220: present), the controlling section 10 proceeds to the process in S240. With respect to the passenger P determined to be not bleeding (S220: absent), the controlling section 10 proceeds to the process in S250.

In S230, the controlling section 10 determines whether or not the heart of the passenger P is beating. The controlling section 10 determines whether or not the heart of the passenger P is beating, for example, on the basis of an inferred value of the heart rate of the passenger P which has been inferred by the inferring section 101. For example, in a case where the inferred value of the heart rate of the passenger P inferred by the inferring section 101 is not more than a predetermined threshold and not less than 10 minutes have elapsed from the timing at which the collision accident occurred (S230: absent for 10 minutes), the controlling section 10 determines that the urgency level of the injury of the passenger P is “black: 0 (the most urgent)” (S301). Here, the predetermined threshold is, for example, a value less than 100 times/minute. In a case where the inferred value of the heart rate of the passenger P inferred by the inferring section 101 is larger than the predetermined threshold or in a case where the inferred value of the heart rate of the passenger P inferred by the inferring section 101 is not more than the predetermined threshold and less than 10 minutes have elapsed from the timing at which the collision accident occurred (S230: present, or absent for less than 10 minutes), the controlling section 10 determines that the urgency level of the injury of the passenger P is “red: I (urgent)” (S302).

In S240, in a case where the heart rate and the respiratory rate of the passenger P correspond to the level 1 or 2 in conformity with the indices shown in FIG. 4 (S240: corresponding to level 1, 2), the controlling section 10 determines that the urgency level of the injury of the passenger P is “yellow: II (semi-urgent)!” (S303). In a case where the heart rate and the respiratory rate of the passenger P correspond to the level 3 or 4 in conformity with the indices shown in FIG. 4 (S240: corresponding to level 3, 4), the controlling section 10 determines that the urgency level of the injury of the passenger P is “red: I (urgent)” (S304).

In S250, in a case where the respiratory rate of the passenger P is not less than 10 times/minute and less than 30 times/minute, the controlling section 10 proceeds to the process in S260, and in a case where the respiratory rate of the passenger P is not less than 30 times/minute or less than 10 times/minute, the controlling section 10 determines that the urgency level of the injury of the passenger P is “red: I (urgent)” (S304).

In S260, in a case where the heart rate of the passenger P is less than 120 times/minute, the controlling section 10 proceeds to the process in S270, and in a case where the heart rate of the passenger P is not less than 120 times/minute or in a case where it is impossible to touch the passenger P, the controlling section 10 determines that the urgency level of the injury of the passenger P is “red: I (urgent)” (S304). Here, the case where it is impossible to touch the passenger P is, for example, a case where the detection signal from the sensor 20 is not more than a third threshold. The third threshold is smaller than the first threshold Vth.

In S270, the controlling section 10 makes determination as to a consciousness level of the passenger P. In a case where the passenger P does not respond to an easy instruction, the controlling section 10 determines that the urgency level of the injury of the passenger P is “red: I (urgent)” (S304), and in a case where the passenger P responds to the easy instruction, the controlling section 10 determines that the urgency level of the injury of the passenger P is “yellow: II (semi-urgent)” (S305). Here, the case where the passenger P does not respond to an easy instruction is, for example, a case where the passenger P does not carry out a predetermined operation in accordance with a voice instruction via a speaker or the like within a predetermined time. The predetermined operation is, for example, an operation for notifying the occurrence of an accident.

### Embodiment 2

The following description will discuss Embodiment 2 of the present disclosure. Note that, for convenience of description, an identical reference numeral will be given to a member having a function identical to that of a member described in Embodiment 1, and description of the member will be omitted.

The information processing device 1 in accordance with Embodiment 2 of the present disclosure differs from that in accordance with Embodiment 1 above in a method for inferring a bleeding volume of the passenger P with use of the inferring section 101. In the information processing device 1 in accordance with Embodiment 2 of the present disclosure, the inferring section 101 infers a seating pressure applied from the passenger P to the seat 2A or the like, on the basis of the detection signal obtained by the obtaining section 100, and infers a bleeding volume of the passenger P on the basis of a change in a result of the inference of the seating pressure before and after the determining section 102 determines that the movable body 200 has been involved in a collision accident.

It has been known that there are individual differences in whether or not tachycardia occurs when a body bleeds in a large amount. Inferring a bleeding volume of a passenger P on the basis of a change in the inference result of the seating pressure makes it possible to also appropriately infer a bleeding volume of a passenger P who does not suffer from tachycardia when bleeding in a large amount.

In the information processing device 1 in accordance with Embodiment 2 of the present disclosure, it is also possible that the passenger P is caused to input the body weight in advance at any timing before the accident occurs, e.g., the timing of boarding on the movable body 200, so that the process of inferring the seating pressure before the occurrence of an accident is omitted.

### Embodiment 3

The following description will discuss Embodiment 3 of the present disclosure. Note that, for convenience of description, an identical reference numeral will be given to a member having a function identical to that of a member described in Embodiments 1 and 2 above, and description of the member will be omitted.

The information processing device 1 in accordance with Embodiment 3 of the present disclosure differs from those in accordance with Embodiments 1 and 2 above in a method for inferring a bleeding volume of the passenger P with use of the inferring section 101. In the information processing device 1 in accordance with Embodiment 3 of the present disclosure, the inferring section 101 infers a cardiac sound of the passenger P on the basis of the detection signal obtained by the obtaining section 100, and infers a bleeding volume of the passenger on the basis of a change in a result of the inference of the cardiac sound before and after the determining section 102 determines that the movable body 200 has been involved in a collision accident.

In a scene of an emergency medical service, the Focused Assessment with Sonography for Trauma (FAST) may be used for checking the presence or absence of bleeding. A piezoelectric sensor is a sensor that generates a voltage in response to external vibration, and is sometimes used as, for example, an ultrasonic probe in a FAST. In Embodiment 3 of the present disclosure, the sensor 20 is a piezoelectric sensor and detects a cardiac sound of the heart of the passenger P, which is an ultrasonic sound source. The inferring section 101 infers a cardiac sound of the passenger P on the basis of the detection signal from the sensor 20. The inferring section 101 may infer the bleeding volume on the basis of the cardiac sound of the passenger P inferred before and after the accident.

(Effects) As described above, the information processing device 1 in accordance with the present embodiments can provide the following effects.

The information processing device 1 includes: the obtaining section 100 that obtains a detection signal from the sensor 20 that is disposed in the seat 2A or the like of the movable body 200 and that detects vibration transmitted from the passenger P seated on the seat 2A or the like to the seat 2A or the like; and the inferring section 101 that infers a bleeding volume of the passenger P on the basis of the detection signal obtained by the obtaining section 100. The above configuration makes it possible to appropriately infer a bleeding volume of the passenger P of the movable body 200.

The inferring section 101 infers a heart rate and a respiratory rate of the passenger P on the basis of the detection signal obtained by the obtaining section 100 and infers a bleeding volume of the passenger P on the basis of a result of the inference of the heart rate and the respiratory rate of the passenger P. In a case where internal bleeding in a large amount occurs, such as a case where the pelvis of the passenger P is fractured, it is difficult to infer a bleeding volume from an image of an appearance of the passenger imaged by, for example, a camera. According to the above configuration, the bleeding volume of the passenger P is inferred on the basis of the heart rate and the respiratory rate of the passenger P, so that it is possible to appropriately infer the bleeding volume of the passenger P, even if there is much internal bleeding.

The inferring section 101 further infers an urgency level of injury of the passenger P on the basis of a result of inference of the bleeding volume, the heart rate, and the respiratory rate of the passenger P. The information processing device 1 further includes the notifying section 103 that notifies the urgency level of the injury of the passenger P to an inside and outside of the movable body 200. According to the above configuration, notification of the urgency level of the injury of the passenger P is made on the basis of the inference result of the bleeding volume, the heart rate, and the respiratory rate of the passenger P, so that it is possible to promote emergency call and life-saving actions around the accident site.

The information processing device 1 further includes the determining section 102 that determines whether or not the movable body 200 has been involved in a collision accident, on the basis of the detection signal obtained by the obtaining section 100. The inferring section 101 infers a seating pressure applied from the passenger P to the seat 2A or the like, on the basis of the detection signal obtained by the obtaining section 100, and infers a bleeding volume of the passenger P on the basis of a change in a result of the inference of the seating pressure before and after the determining section 102 determines that the movable body 200 has been involved in a collision accident. It has been known that there are individual differences in whether or not tachycardia occurs when a body bleeds in a large amount. According to the above configuration, inferring a bleeding volume of a passenger P on the basis of a change in the inference result of the seating pressure makes it possible to also appropriately infer a bleeding volume of a passenger P who does not suffer from tachycardia when bleeding in a large amount.

The information processing device 1 further includes the determining section 102 that determines whether or not the movable body 200 has been involved in a collision accident, on the basis of the detection signal obtained by the obtaining section 100. The inferring section 101 infers a cardiac sound of the passenger P on the basis of the detection signal obtained by the obtaining section 100, and infers a bleeding volume of the passenger P on the basis of a change in a result of the inference of the cardiac sound before and after the determining section 102 determines that the movable body 200 has been involved in a collision accident. The above configuration makes it possible to appropriately infer a bleeding volume of the passenger P of the movable body 200.

The movable body 200 includes: the information processing device 1; the sensor 20; the main power source 3 that supplies electric power to the sensor 20 and the information processing device 1; and the spare power source 4 that supplies electric power to the sensor 20 and the information processing device 1 in a case where the main power source 3 malfunctions. The above configuration makes it possible to continue the processes of, for example, inferring a bleeding volume of the passenger P of the movable body 200 after the movable body 200 has been involved in a collision accident.

### Software Implementation Example

The functions of the information processing device 1 (hereinafter, referred to as “device”) can be realized by a program for causing a computer to function as the device, the program causing the computer to function as the control blocks of the device (in particular, the components included in the controlling section 10).

In this case, the device includes, as hardware for executing the program, a computer which includes at least one controlling device (e.g., processor) and at least one storage device (e.g., memory). Each function described in the foregoing embodiments can be realized by the controlling device and the storage device executing the program.

The program may be stored in one or more non-transitory computer-readable recording mediums. This recording medium may or may not be provided in the device. In the latter case, the program can be supplied to or made available to the device via any transmission medium such as a wired transmission medium or a wireless transmission medium.

Further, some or all of functions of the control blocks can be realized by a logic circuit. For example, the present disclosure encompasses, in its scope, an integrated circuit in which a logic circuit that functions as each of the control blocks is formed. As another alternative, for example, it is possible to realize the functions of the control blocks by a quantum computer.

Further, each of processes which are described in the foregoing embodiments may be carried out by artificial intelligence (AI). In this case, the AI may be operated by the controlling device, or alternatively operated by another device (e.g., an edge computer, a cloud server).

### Variations

Although in Embodiments 1 to 3 above, the information processing device 1 is provided in an automobile, the same effects as described above can be exerted also in a case where the information processing device 1 is provided in a movable body other than an automobile. For example, the information processing device 1 may be provided in a movable body such as a bus, a taxi, a railway vehicle, an aircraft, or a ship.

In Embodiments 1 to 3 above, the sensor 20 is a piezoelectric sensor. However, any sensor can be used as the sensor 20, provided that it is a sensor capable of detecting the vibration transmitted from the passenger P seated on the seat 2A or the like to the seat 2A or the like.

In Embodiments 1 to 3 above, one sensor 20 is disposed in a position closer to the left than the center of the seating face in each seating position of the seat 2A and the like. However, the position and the number of the sensors 20 that are disposed in each seating position are not limited to those described in the above embodiments. For example, the sensor 20 may be disposed in a position closer to the right than the center of the seating face, or the sensor 20 may be disposed in a seat back.

In Embodiments 1 to 3 above, FIG. 4 and FIG. 7 show an example of various thresholds used by the inferring section 101 for inferring a bleeding volume and an urgency level of the injury of the passenger P. However, these thresholds are not limited to those illustrated in FIGS. 4 and 7.

Each inference by the inferring section 101 may be carried out with use of a learning model generated by supervised learning in which data stored by the storing section 104 and the result determined for the passenger P by the crew of the emergency vehicle or the doctor is used as training data. In a case where the movable body 200 has been involved in an accident, new training data including the data stored by the storing section 104 of the information processing device 1 provided in the movable body 200 and the result of the determination for the passenger P by the crew of the emergency vehicle or the doctor may be collected in, for example, a server provided outside the movable body 200. The server may update the learning model on the basis of the new collected training data.

The timing at which a doctor makes determination is later than the timing at which a crew of an emergency vehicle makes determination due to, for example, a time for transporting the passenger P. In learning using training data including a result of determination by a doctor, when updating various thresholds used by the inferring section 101 to infer a bleeding volume and an urgency level of injury of the passenger P, it is possible to consider changes over time due to a time for transportation to a hospital, changes in conditions during transportation of the passenger according to a survival curve, and treatment procedures carried out by a crew of an emergency vehicle during transportation. Examples of the survival curve include the Drinker's survival curve, the Cowley's survival curve, and the survival curve indicating life-saving effects exhibited in a case where an emergency medical care is carried out by a bystander.

The various thresholds used by the inferring section 101 to infer a bleeding volume and an urgency level of injury of a passenger P may be corrected on the basis of a result of inference that has been made for the passenger P before the determining section 102 determines that the movable body 200 has been involved in a collision accident. This makes it possible to appropriately infer an urgency level also for a passenger P who usually have a high heart rate or a high respiratory rate.

### ADDITIONAL REMARK

The present disclosure is not limited to the embodiments above, but can be altered in various ways by a skilled person in the art within the scope of the claims. The present disclosure also encompasses, in its technical scope, any embodiment derived by combining technical means disclosed in differing embodiments as appropriate.

### REFERENCE SIGNS LIST

1 Information processing device
2A, 2B, 2C Seat
3 Main power source
4 Spare power source
5 Notifying device
10 Controlling section
20 Sensor
100 Obtaining section
101 Inferring section
102 Determining section
103 Notifying section
104 Storing section
200 Movable body

1. An information processing device comprising:
an obtaining section that obtains a detection signal from a sensor which is disposed in a seat of a movable body and which detects vibration transmitted to the seat from a passenger seated on the seat; and
an inferring section that infers a bleeding volume of the passenger on the basis of the detection signal obtained by the obtaining section.

2. The information processing device according to claim 1, wherein the inferring section infers a heart rate and a respiratory rate of the passenger on the basis of the detection signal obtained by the obtaining section and infers the bleeding volume of the passenger on the basis of a result of inference of the heart rate and the respiratory rate of the passenger.

3. The information processing device according to claim 2, wherein the inferring section further infers an urgency level of injury of the passenger on the basis of a result of inference of the bleeding volume, the heart rate, and the respiratory rate of the passenger, and
the information processing device further comprises a notifying section that notifies the urgency level of the injury of the passenger to an inside and outside of the movable body.

4. The information processing device according to claim 1, further comprising a determining section that determines whether or not the movable body has been involved in a collision accident, on the basis of the detection signal obtained by the obtaining section,
the inferring section inferring a seating pressure applied from the passenger to the seat, on the basis of the detection signal obtained by the obtaining section and inferring the bleeding volume of the passenger on the basis of a change in a result of inference of the seating pressure before and after the determining section determines that the movable body has been involved in a collision accident.

5. The information processing device according to claim 1, further comprising a determining section that determines whether or not the movable body has been involved in a collision accident, on the basis of the detection signal obtained by the obtaining section,
the inferring section inferring a cardiac sound of the passenger on the basis of the detection signal obtained by the obtaining section and inferring the bleeding volume of the passenger on the basis of a change in a result of inference of the cardiac sound before and after the determining section determines that the movable body has been involved in a collision accident.

6. A movable body comprising:
the information processing device according to claim 1;
the sensor;
a first power source that supplies electric power to the sensor and the information processing device; and
a second power source that supplies electric power to the sensor and the information processing device in a case where the first power source malfunctions.

7. A non-transitory computer-readable recording medium that stores a program for causing a computer to function as the information processing device according to claim 1 and that stores the program for causing the computer to function as the obtaining section and the inferring section.
